(19) United States
(12) Patent Application Publication
(10) Pub. No.: US 2021/0228488 A1
(43) Pub. Date: Jul. 29, 2021

(54) EXTENDED RELEASE COMPOSITIONS AND PROCESS FOR PREPARATION

(71) Applicant: RUBICON RESEARCH PRIVATE LIMITED, Thane West (IN)

(72) Inventors: Pratibha Pilgaonkar, Thane West (IN); Jayant Karajgi, Thane West (IN); Shubhrangshu Sarkar, Thane West (IN); Anusha Palani, Thane West (IN)

(21) Appl. No.: 17/051,047

(22) PCT Filed: Apr. 27, 2019
(86) PCT No.: PCT/IN2019/050341

§ 371 Date: Oct. 27, 2020

(30) Foreign Application Priority Data
Apr. 27, 2018 (IN) 201721038751

(51) Int. Cl. A61K 9/20; A61K 31/138; A61K 31/554; A61K 45/06

(52) U.S. Cl. CPC A61K 9/2054; A61K 9/2031; A61K 9/2027; A61K 9/2013; A61K 45/06; A61K 31/138; A61K 31/554; A61K 9/2095; A61K 9/2009

(57) ABSTRACT

The present invention relates to multiple unit extended release pharmaceutical compositions comprising plurality of modified release units wherein each unit comprises of an active agent core comprising at least one pharmaceutically active agent and at least one pharmaceutically acceptable excipient substantially coated with at least one release-controlling agent. The present invention also relates to a process for the preparation of these multiple unit extended release pharmaceutical compositions.

### FIELD OF THE INVENTION

The present invention relates to multiple unit, extended release pharmaceutical compositions and the process for their preparation. Particularly the extended release compositions comprise plurality of modified release units, with each unit comprising an active agent core substantially coated with at least one release controlling agent. The active agent core comprises at least one pharmaceutically active agent and at least one pharmaceutically acceptable excipient. The invention further relates to preparation of such extended release formulations in the form of tablets, capsules, mini-tablets, orally disintegrating tablets, granules, dispersible tablets, and the like.

### BACKGROUND OF THE INVENTION

The concept of extended release formulations was developed to reduce the number of daily drug administrations, particularly for those drugs requiring reasonably constant blood levels over a long period of time and to improve patient compliance. Extended release compositions have also been designed to reduce incidences of adverse drug reactions by providing reduced fluctuations in the concentrations of the drug in the plasma. Extended release formulations have been adopted for those drugs that need to be administered at high doses, but are likely to cause undesirable side effects by a fast release of the drug.

Amongst various formulation approaches available towards extending the release of active agents, the development of multiple unit systems in which each individual unit is formulated with modified release characteristics, has unique advantages. Multiple unit dosage forms possess a large surface area, which advantageously promotes complete and uniform absorption, minimizes peak plasma fluctuations and thus reduces the potential for systemic side effects. A further advantage of these dosage forms is that high local concentrations of the active substance in the gastrointestinal system is avoided as a consequence of the units being distributed freely throughout the tract. Additionally, the chances of dose dumping likely with certain actives and systems is also minimized with the multiple unit systems. Hence, the multiple unit dosage form ensures appropriate release of the active agent, resulting in a decreased dosing frequency and consequently better patient compliance. Formulation of drugs in multiple-unit dosage forms with units filled in capsules or compressed into tablets, offers flexibility to provide desired drug release properties.

Multiple unit systems, also referred to as multiple unit pellet systems (MUPS) are usually prepared by the process of drug layering wherein the modified release units are prepared by coating inert beads or spheres, followed by coating with release rate controlling polymers. Numerous marketed products are based on this approach. Some examples include, metoprolol succinate (Toprol-XL® tablet), cyclobenzaprine hydrochloride (Amrix® E.R. capsules), fluvoxamine maleate (Luvox® E.R. capsule); tolterodine tartrate (Detrol® LA E.R. capsule).

The currently marketed extended release dosage form of metoprolol succinate Toprol-XL® tablet, for example, a multiparticulate tablet dosage form comprises silicon dioxide beads as an inert core coated with active agent and release rate controlling polymers. Various patents and patent applications have also been filed for extended release formulations of metoprolol succinate based on this layering concept. U.S. Pat. No. 8,815,285 discloses an extended dosage form of metoprolol or a salt comprising an inert core, wherein inert core is coated with a drug coat comprising metoprolol or a salt thereof and optionally other pharmaceutically acceptable excipients, said drug coat optionally being further coated with one or more pharmaceutically acceptable release rate-controlling polymers. U.S. Pat. No. 4,957,745 discloses controlled release preparation containing a number of beads comprising a salt of metoprolol as the main soluble component, and a method for the production thereof. The beads may contain metoprolol alone or may consist of insoluble cores coated with metoprolol. Examples of insoluble cores are silicon dioxide and small particles of glass. U.S. Patent Application No. 20070202172 discloses an extended release tablet comprising metoprolol succinate pellets and pharmaceutically acceptable excipients, each pellet comprising an inert core, a drug layer and a rate controlling film coating. U.S. Pat. No. 4,927,640 describes controlled release beads having glass or silicon dioxide core, metoprolol succinate sprayed on to the cores of silicon dioxide, glass or sodium chloride from a solution of ethanol 95% and methylene chloride. Then, the coated beads are filled into hard gelatin capsules.

The drug layering approach however requires specialized equipment and is time consuming. It also results in batch to batch assay variations and uniformity issues. Drug layering approach is also associated with long production cycle times and scale-up concerns due to the complex and tedious nature of the drug layering process. Further with the conventional drug layering approach, large amounts of cushioning granules are often required to compress the coated drug layered pellets without causing any rupturing of the release controlling coats. This not only results in increased tablet weight and size but could also cause potential blend segregation during the manufacturing operations due to differences in the physical nature of the conventional drug layered pellets and cushioning granules.

A need therefore exists to have a multiple unit system that is simple, robust, economical and less time consuming to manufacture. Such a system should not require specialized equipment and should provide greater assurance of uniformity, scalability and commercial viability. Such a system should also reduce batch to batch variability while providing the desired extended drug release profile.

The present inventors after excessive efforts and experimentation have developed the extended release pharmaceutical formulations that address such a need and eliminate the drawbacks associated with the conventional multiple unit systems involving drug layering. The present invention provides multiple unit extended release system that avoids the tedious and time-consuming step of drug layering and thereby provides formulations with reduced batch to batch assay variations. The systems of the present invention prevent process loss of active and minimize the production cycle times when compared to the conventional drug layering based systems. The present inventors have also addressed the issues associated with rupture of coatings resulting in dissolution failures or dose dumping and need for high amounts of cushioning granules to avoid the same.

The present inventors provide multiple unit, extended release pharmaceutical compositions comprising plurality of modified release units with each unit comprising an active agent core substantially coated with at least one release controlling agent. The active agent core comprises at least one pharmaceutically active agent and at least one pharmaceutically acceptable excipient. In one aspect, the active agent cores substantially coated with at least one release-controlling agent are further coated with a cushioning top coat. In another aspect, the active agent in the active agent cores of the present invention is not layered on any inert particle. The formulations of present invention provide release of the active agent at a predetermined rate thereby avoiding any undesired rapid release of the drug which can lead to undesirable side effects while also avoiding incomplete release of the drug from the sustained release pharmaceutical formulation. The formulations of the present invention are simple, economical, less time consuming and do not require any specialized equipment. The use of commercially available excipients and reduced production cycle times further allows for the reduction of production costs. The present invention provides for minimized batch to batch variations and reduction in the likelihood of rupture of the release-controlling coat otherwise observed with conventional multiple unit systems (MUPS). Furthermore, the formulations of the present invention can be presented in the form of tablets, capsules, mini tablets, orally disintegrating tablets, granules, dispersible tablets, and the like. The present invention provides compositions and method of preparation thereof.

### SUMMARY OF THE INVENTION

The present invention relates to multiple unit extended release pharmaceutical compositions comprising plurality of modified release units wherein each unit comprises an active agent core substantially coated with at least one release controlling agent. In one aspect, the active agent core comprises at least one pharmaceutically active agent and at least one pharmaceutically acceptable excipient. In another aspect, the active agent core substantially coated with at least one release-controlling agent is further coated with a cushioning top coat. The present invention also relates to a process for the preparation of these multiple unit extended release pharmaceutical compositions.

### DETAILED DESCRIPTION OF THE INVENTION

The present invention relates to multiple unit, extended release pharmaceutical compositions and the process for their preparation. Particularly the extended release compositions comprise plurality of modified release units, with each unit comprising an active agent core substantially coated with at least one release controlling agent. The active agent core comprises at least one pharmaceutically active agent and at least one pharmaceutically acceptable excipient. In one aspect, the active agent in the active agent cores of the present invention is not layered on any inert particle. In another aspect the active agent core substantially coated with at least one release-controlling agent is further coated with a cushioning top coat.

The term “composition” or “formulation” or “dosage form” or “preparation” has been employed interchangeably for the purpose of the present invention and mean that it is a pharmaceutical formulation which is suitable for administration to a patient. For the purpose of the present invention, the terms “controlled release” or “sustained release” or “extended release” or “modified release” or “prolonged release” have been used interchangeably and mean broadly that the active agent is released at a predetermined rate that is different or slower than immediate release.

The extended release compositions of the present invention comprise plurality of modified release units, with each unit comprising an active agent core substantially coated with at least one release controlling agent. The active agent core comprises at least one pharmaceutically active agent and at least one pharmaceutically acceptable excipient.

The term “pharmaceutically active agent/s” as employed herein refers to any suitable drug for which extended release is desired. In one embodiment, active agents having different solubilities in water may be delivered by the compositions of the present invention. In a further embodiment, the formulation of the present invention can be used to deliver pharmaceutically active agents having drug solubility such as, but not limited to, very soluble solubility, freely soluble solubility, soluble solubility, sparingly soluble solubility, slightly soluble solubility, very slightly soluble active agents. In a further embodiment, the formulation of the present invention can be used to deliver pharmaceutically active agents that are, but not limited to, very soluble, freely soluble, soluble, sparingly soluble, slightly soluble, very slightly soluble active agents. In another embodiment, the formulation of the present invention can be used to deliver pharmaceutically active agents with low solubility. In a further embodiment, the formulation of the present invention can be used to deliver active agents with high solubility. In a further embodiment, pharmaceutically active agents having solubility falling in any solubility range can be delivered by the formulations of the present invention.

In one embodiment, the formulation of the present invention can be used to deliver different dose ranges of the pharmaceutically active agent. In another embodiment, the formulation of the present invention can be used to deliver high dose pharmaceutically active agent. In a further embodiment, the formulation of the present invention can be used to deliver low dose pharmaceutically active agent. In one embodiment, pharmaceutically active agents having high solubility and high dose can be delivered by the formulations of the present invention. In another embodiment, pharmaceutically active agents having high solubility and low dose can be delivered by the formulations of the present invention. In a further embodiment, pharmaceutically active agents having low solubility and high dose can be delivered by the formulations of the present invention. In one embodiment, pharmaceutically active agents having low solubility and low dose can be delivered by the formulations of the present invention.

In general all, including, but not limited to, acidic, basic or amphoteric drugs or any combinations thereof can be incorporated in the compositions of the present invention. Active agents that exhibit a tendency to cause severe side effects when administered frequently in immediate release formulations are also potential candidates in the composition of the present invention.

Pharmaceutically active agents that can be employed in the present invention include, but are not limited to, psychostimulants, antihistamines, expectorants, mucolytics, anti-tussive agents, serotonin reuptake inhibitors, norepinephrine reuptake inhibitors, sympatholytics, antipsychotics, anti-muscarinics, urinary antispasmodics, PDE5 inhibitors, anti-Alzheimer's agents, analgesics, decongestants, analeptic agents, anesthetic agents, anti-asthmatics, anti-arthritic agents, anti-cancer agents, anti-cholinergic agents, anti-convulsant agents, anti-depressant agents, anti-diabetics, anti-helminthic agents, anti-diarrheal agents, anti-epileptics, anti-hyperlipidemic agents, anti-hypertensive agents, anti-hypotensive agents, anti-infective agents, anti-inflammatory agents, non-steroidal anti-inflammatory agents, anti-emetics, anti-migraine agents, anti-neoplastic agents, anti-tubercular agents, antibiotics, antacids, antiulcer agents, anti-Parkinsonism drugs, anti-pruritic agents, anti-pyretic agents, anti-spasmodics, anti-viral agents, anxiolytic agents, appetite suppressants, attention deficit hyperactivity disorders agents, cardiovascular agents, calcium channel blockers, anti-anginal agents, central nervous system agents, beta-blockers, antiarrhythmic agents, bronchodilators, central nervous system stimulants, diuretics, genetic materials, hormonolytics; hypnotics, hypercalcemics, hypoglycemic agents, immunosuppressive agents, beta-agonists, narcotic antagonists, nicotine, nutritional agents, parasympatholytics, peptide drugs, anti-hemorrhoidals, psychotropics, mucolytics, sedatives, laxatives, vitamins, sialagogues, steroids, sympathomimetics, tranquilizers, vasodilators, or any combinations thereof.

In one embodiment, pharmaceutically active agents that can be employed in the present invention include, but are not limited to, amphetamine, amphetaminil, atomoxetine, dexmethylphenidate, dextroamphetamine, dextromethamphetamine, cyclobenzaprine, propranolol, fencamfamine, fenethylline, lisdexamfetamine, methylphenidate, mesocarb pemoline, pipradrol, prolintane, dimenhydrinate, diphenhydramine, chlorpheniramine, brompheniramine, dexchlorpheniramine, hydroxyzine, dexbrompheniramine, fexofenadine, terfenadine, cetirizine, levocetirizine, ambroxol, bromhexine, carbocisteine, domiodol, guaifenesin, codeine, dextromethorphan, hydrocodone, clovoxamine, desvenlafaxine, naltrexone, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline, zimelidine, clonidine, guanfacine, methyldopa, iloperidone, ocaperidone, paliperidone, risperidone, lurasidone, perospirone, revospirone, tiospirone, ziprasidone, darifenacin, emepronium, fesoterodine, flavoxate, imidafenacin, meladrazine, mirabegron, oxybutynin, propiverine, solifenacin, terodiline, esomeprazole, omeprazole, pantoprazole, lansoprazole, dexlansoprazole, tolterodine, trospium chloride, acetildenafil, aildenafil, avanafil, icariin, lodenafil, mirodenafil, nitrosoprodenafil, sildenafil, sulfoaildenafil, tadalafil, udenafil, vardenafil, memantine, neramexane (1, 3, 3, 5, 5-pentamethylcyclohexan-1-amine), donepezil, tacrine, rivastigmine, galantamine, physostigmine, neostigmine, Huperzine A, icopezil (CP-118954; 5,7-dihydro-3-[2-[1-(phenylmethyl)-4-piperidinyl]ethyl]-6H-pyrrolo-[4,5-f]-1,2-benzisoxazol-6-onemaleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl) piperidin-4-yl]-1-(2,3,4,5-tetrahydro-1H-1-benzazepin-8-yl)-1-propane fumarate), metrifonate (T-588; (-)-R-α-[[2-(dimethylamino)ethoxy]methyl] benzo [b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f] oxepine-10-methanamine), SDZ-220-581 ((S)-α-amino-5-(phosphonomethyl)-[1,1′-biphenyl]-3-propionic acid), tarenflurbil, tramiprosate, clioquinol, aspirin, codeine, morphine, dihydromorphone, oxycodone, hydrocodone, phenylephrine, pseudoephedrine, theophylline, phenobarbital sodium, phenytoin sodium, valproate sodium barbiturates, amylobarbitone sodium, butabarbital sodium, secobarbital sodium, metformin; phenytoin, meprobamate, nitrezepam, oxcarbazepine, methyldopa; captopril, naproxen, diclofenac, indomethacin, ibuprofen, sulindac, meclofenamate sodium, tolmetin sodium, metoclopramide, tetracyclines, atropine, scopolamine, albuterol, ethacrynic acid, bendrofluazide, nifedipine, papaverine, diltiazem, or nicardirine or any combinations thereof.

In one embodiment, the active agent/s employed in the present invention may be in the form of free base or acid or pharmaceutically acceptable salts, prodrugs, active metabolites, polymorphs, solvates, hydrates, enantiomers, optical isomers, tautomers or racemic mixtures thereof. Pharmaceutically effective amount of active agent is employed in the composition of the present invention. The term “effective amount” refers to an amount effective to achieve desired preventive, therapeutic and/or beneficial effect. In one embodiment the amount of pharmaceutically active agent in the extended release compositions can vary from about 5% by weight to about 95% by weight. In another embodiment the amount of pharmaceutically active agent in the extended release compositions can vary from about 10% by weight to about 90% by weight. In still another embodiment, the amount of pharmaceutically active agent in the extended release compositions can vary from about 15% by weight to about 85% by weight, based on the total weight of the compositions.

The active agent core further comprises at least one pharmaceutically acceptable excipient, such as, but not limited to, diluents, binders, glidants, lubricants or anti-adherents and the like or any combinations thereof. Suitable diluents that may be employed include, but are not limited to, starch, talc, microcrystalline cellulose, lactose, xylitol, mannitol, maltose, polyols, fructose, sorbitol, magnesium hydroxide, dicalcium phosphate, and the like or any combinations thereof. Suitable binders that may be employed include, but are not limited to, starch, pregelatinized starch, polyvinyl pyrrolidone, copovidone, cellulose derivatives, such as hydroxypropylmethyl cellulose, hydroxypropyl cellulose and carboxymethyl cellulose and their salts, and the like or any combinations thereof. Suitable lubricants or anti-adherents that may be employed include, but are not limited to, magnesium stearate, calcium stearate, stearic acid, talc, sodium stearyl fumarate, and the like or any combinations thereof. Suitable glidants that may be employed include but are not limited to, silica gel, colloidal silicon dioxide, talc, silica, and the like or any combinations thereof.

In one embodiment, the active agent cores of the present invention are in the form of granules prepared according to the methods such as, but not limited to, dry granulation, roller compaction, slugging or the combinations thereof. In a further embodiment, the active agent cores of the present invention are in the form of compacted granules.

In one embodiment the active agent cores are prepared by a process of roll compaction and milling. In a further embodiment, the active agent cores of the present invention may range in size from about 1 micrometer to about 1000 micrometers. In another embodiment, the active agent cores of the present invention may range in size from about 1 micrometer to about 750 micrometers.

In one embodiment, the active agent cores prepared by the process of the present invention maintain their integrity during the further steps of coating. In another embodiment, the process of preparation of the active agent cores of the present invention provides a good yield of the active agent cores of consistent friability.

In another embodiment, the active agent cores of the present invention may be present in the compositions of the present invention in an amount from about 5% to about 90% by weight of the composition. In a further embodiment, the active agent cores may comprise the active agent in an amount of greater than 60%. In another embodiment, the active agent cores may comprise the active agent in an amount of greater than 70%. In another embodiment, the active agent cores may comprise the active agent in an amount of greater than 75%. In another embodiment, the active agent cores may comprise the active agent in an amount of about 80%. In a further embodiment, the active agent cores may comprise the active agent in an amount of greater than 80%.

In a further embodiment, extended release pharmaceutical compositions of the present invention comprise active agent cores substantially coated with at least one release controlling agent. In a further embodiment, active agent cores in the form of compacted granules comprising at least one pharmaceutically active agent are coated with at least one release controlling agent. In one embodiment, the release controlling agent used may be polymeric or non-polymeric or the mixtures thereof. In a further embodiment, the release controlling polymeric agent may be pH-dependent or pH-independent in nature or combinations thereof. In another embodiment, the extended release polymeric agent may be pH-independent in nature.

In another embodiment, the polymeric release controlling agents that may be employed in the compositions of the present invention include, but are not limited to, cellulose derivatives, acrylic acid derivatives, maleic acid derivatives, polymers and copolymers, vinyl derivatives, polymers and copolymers and the like or combinations thereof. Cellulose derivatives that may be employed in the present invention include, but are not limited to, ethyl cellulose, hydroxypropylmethyl cellulose, hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, carboxymethylethylcellulose, ethylhydroxyethylcellulose phthalate and the like or combinations thereof. Acrylic acid derivatives include, but are not limited to, styrene⋅acrylic acid copolymer, methyl acrylate⋅methacrylic acid copolymer, butyl acrylate⋅styrene⋅acrylic acid copolymer, methacrylic acid polymers and copolymers, polyacrylates, methacrylate polymers and copolymers such as, but not limited to, a) copolymer formed from monomers selected from methacrylic acid, methacrylic acid esters, acrylic acid and acrylic acid esters b) copolymer formed from monomers selected from butyl methacrylate, (2-dimethylaminoethyl)methacrylate and methyl methacrylate c) copolymer formed from monomers selected from ethyl acrylate, methyl methacrylate and trimethylammonioethyl methacrylate chloride or d) copolymers of acrylate and methacrylates with/without quarternary ammonium group in combination with sodium carboxymethylcellulose, e.g. those available from Rohm GmbH under the trademark Eudragit® like Eudragit® EPO (dimethylaminoethyl methacrylate copolymer; basic butylated methacrylate copolymer), Eudragit® RL and RS (trimethylammonioethyl methacrylate copolymer), Eudragit® NE30D and Eudragit® NE400 (ethylacrylate methymethacrylate copolymer), Eudragit® L 100 and Eudragit® S (methacrylic acid.methyl methacrylate copolymer), Eudragit® L100-55 (methacrylic acid.ethyl acrylate copolymer); or the like or any combinations thereof. Maleic acid derivatives, polymers and copolymers that may be employed in the present invention include, but are not limited to, vinylacetate maleic acid anhydride copolymer, styrene⋅maleic acid anhydride copolymer, styrene maleic acid monoester copolymer, vinylmethylether maleic acid anhydride copolymer, ethylene maleic acid anhydride copolymer, vinylbutylether maleic acid anhydride copolymer, acrylonitrile methyl acrylate maleic acid anhydride copolymer, butyl acrylate styrene maleic acid anhydride copolymer and the like. Vinyl derivatives, polymers and copolymers include, but are not limited to, polyvinylacetate, copolymers of vinyl pyrrolidone, polyvinyl alcohol, copolymers of polyvinyl alcohol, mixture of polyvinyl acetate and polyvinylpyrrolidone (e.g. Kollidon® SR), polyvinyl alcohol phthalate, polyvinylacetal phthalate, polyvinyl butylate phthalate, polyvinylacetoacetal phthalate, or combinations thereof.

In another embodiment, the extended release polymeric agent employed in the present invention is ethyl cellulose or the combinations thereof.

In one embodiment, the non-polymeric release controlling agents that may be employed in the composition of the present invention include, but are not limited to, waxes, hydrogenated vegetable oils and the like. Suitable waxes that may be employed include, but are not limited to, carnauba wax, candelilla wax, spermaceti, bees wax, montan wax, microcrystalline wax, lecithin, paraffin wax, cetyl alcohol, cetostearyl alcohol and the like or combinations thereof. Suitable hydrogenated vegetable oils that may be employed include, but are not limited to, hydrogenated cottonseed oil, hydrogenated soybean oil, compritol and the like or combinations thereof.

In a further embodiment, the release controlling coatings of the present invention may be used in admixture with at least one pharmaceutically acceptable excipient, such as but not limited to, plasticizers, pore-formers, pigments and the like or any mixtures thereof. Suitable plasticizers include, but are not limited to, dibutyl sebacate, propylene glycol, polyethylene glycol, polyvinyl alcohol, triethyl citrate, acetyl triethyl citrate, acetyl tributyl citrate, tributyl citrate, triacetin or the like or any combinations thereof. Suitable pore-formers that may be employed include, but are not limited to, hydroxypropyl methyl cellulose, dextrates and the like or combinations thereof.

A coating procedure known to a person skilled in the art, which substantially coats the active agent cores without significant agglomeration of the cores, may be used. Coatings may be applied in a coating pan or with a fluid-bed coating apparatus. The controlled release coatings may be applied from aqueous suspension or organic solvents. Optimum coat weight and coat thickness may be determined for each type of active cores and generally depends on the drug release characteristics desired for that particular active agent.

The amount of release controlling agent used in the formulation may vary depending upon the pharmaceutically active agent employed and the degree of extended release desired. In one embodiment, the release controlling agent is present in the present invention in an amount from about 0.1% to about 60% by weight. In another embodiment, the release controlling agent is present in the present invention in an amount from about 0.5% to about 55% by weight. In a further embodiment, the release controlling agent is present in the present invention is in an amount from about 1% to about 50% by weight.

In one embodiment, one or more coating layers of same or different release controlling agent can be applied on the active agent cores. In another embodiment, sub-coating of a film forming polymer can be applied on the active agent cores prior to the application of the release controlling coating agent. Suitable polymers for sub-coating can be selected from the list mentioned hereinabove for release controlling agents. In a further embodiment, ethyl acrylate methyl methacrylate copolymer is employed as a polymer for a sub-coat layer. In one aspect, the sub-coating layer is applied in order to smoothen the surface of the active cores for further coating with the release-controlling agent.

In a further embodiment, the active agent cores of the present invention substantially coated with the release controlling agent may be further coated with a cushioning top coat. In one embodiment, the cushioning top coat employed in the present invention may be a hydrophilic polymer such as, but not limited to, polyethylene glycol, and the like or any combinations thereof.

Without being bound to any theory, it is believed that active agent cores prepared by the process of roll compaction and milling provide compacted granules that can be substantially coated with release controlling polymer and when the coated active agent cores are incorporated in the compositions of the present invention they reduce batch to batch assay variability and provide the desired release profile. Without being bound to any theory it is believed that the cushioning top coat aids maintain the integrity of the release-controlling coat and minimizes fracturing of the release-controlling coating layer even after being subjected to stress, including compression during tableting operation.

In one embodiment, the coated active agent cores are the modified release units of the present invention. In a further embodiment, the modified release units of the present invention are the final extended release compositions. In another embodiment, the modified release units of the present invention are blended with one or more pharmaceutically acceptable excipients selected depending on the final dosage form. In yet another embodiment, the modified release units of the present invention are blended with one or more pharmaceutically acceptable excipients or with granules of one or more pharmaceutically acceptable excipients depending on the final dosage form.

In one embodiment, the at least one pharmaceutically acceptable excipient, that may be incorporated in the formulation of the present invention depending on the final dosage form to be prepared include, but are not limited to, binders, disintegrants, superdisintegrants, diluents, salivating agents, surfactants, flavors, sweeteners, colorants, souring agents, viscolizers, glidants, lubricants, solubilizers, stabilizers, suspending agents, preservatives, cosolvents, anti-caking agents, buffers and the like or any combinations thereof. Suitable disintegrants that may be employed include, but are not limited to, crospovidone, calcium silicate and starch. Suitable superdisintegrants that may be employed include, but are not limited to, natural, modified or pregelatinized starch, crospovidone, croscarmellose sodium, sodium starch glycolate, low-substituted hydroxypropyl cellulose. Suitable binders that may be employed include, but are not limited to, starch, pregelatinized starch, polyvinyl pyrrolidone, copovidone, cellulose derivatives, such as hydroxypropylmethyl cellulose, hydroxypropyl cellulose and carboxymethyl cellulose and their salts. Suitable diluents that may be employed include, but are not limited to, starch, microcrystalline cellulose, lactose, xylitol, mannitol, maltose, polyols, fructose, guar gum, sorbitol, magnesium hydroxide, dicalcium phosphate, coprocessed mannitol and calcium silicate and the like or any combinations thereof. Suitable lubricants that may be employed include, but are not limited to, magnesium stearate, calcium stearate, stearic acid, talc, and sodium stearyl fumarate and the like or combinations thereof. Suitable glidants that may be employed include, but are not limited to, colloidal silica, silica gel, precipitated silica, and the like or combinations thereof. Suitable salivating agents that may be employed include, but are not limited to, micronised polyethylene glycol, sodium chloride or precipitated micronised silica. Suitable solubilizers include, but are not limited to cetostearyl alcohol, cholesterol, diethanolamine, ethyl oleate, ethylene glycol, palmitostearate, glycerin, glyceryl monostearate, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, triethanolmine, or combinations thereof.

In a further embodiment, the plurality of modified release units of the present invention are incorporated in the extended release pharmaceutical formulations by any of the methods generally known to a person skilled in the art, especially depending on the final dosage form of the pharmaceutical composition, but not limited to, blending, compression, sifting, milling, filling, physical mixing or the combinations thereof.

In one embodiment, different populations of modified release units exhibiting different release profiles may be incorporated in the extended release pharmaceutical formulations of the present invention. In a further embodiment, the extended release formulations of the present invention can comprise one or more populations of modified release units to provide the desired release profile of the pharmaceutically active agent. In another embodiment, the extended release formulations of the present invention can comprise one or more populations of modified release units prepared using different release controlling agents or combinations thereof.

In one embodiment, the extended release compositions are for oral delivery. The compositions for oral delivery may be in any form, such as, but not limited to, liquid, solid or semisolid preparations and the like. In a further embodiment, the extended release preparation of the present invention is solid preparations. Solid preparations for oral administration may be in any form including, but not limited to core tablets, film coated tablets, capsules, mini tablets, orally disintegrating tablets, granules, powders, dispersible tablets, and the like or any combinations thereof. In a further embodiment, the modified release preparation of the present invention is a film coated tablet. In one aspect, film coating polymers commonly used in the pharmaceutical art may be employed to prepare the formulations of the present invention. In one embodiment, the tablets may be prepared by compression. In another embodiment, the extended release preparation of the present invention is a capsule.

In one embodiment, the present invention also relates to a process for preparing an extended release multiple unit dosage form. In one embodiment, the process of preparation comprises (a) dry granulating/compacting and milling pharmaceutically active agent and at least one pharmaceutically acceptable excipient to form dry granules or compacted granules (b) screening the compacted/dry granulated material to provide uniform sized active agent cores (c) coating the active agent cores with at least one release controlling polymer (d) blending and lubrication of coated active agent cores with at least one pharmaceutically acceptable excipient, and (d) compression of blend to produce the extended release dosage forms, optionally, coating of extended release dosage forms.

In another embodiment, the process of preparation comprises (a) dry granulating/compacting and milling pharmaceutically active agent and at least one pharmaceutically acceptable excipient to form dry granules or compacted granules (b) screening the compacted/dry granulated material to provide uniform sized active agent cores (c) coating the sized active agent cores with a sub-coating layer (d) coating the sub-coated active agent cores with at least one release controlling polymer (e) coating the active agent cores with at least one cushioning top coat, (f) blending and lubrication of coated active agent cores with at least one pharmaceutical acceptable active agent, and (d) compression of the blend to produce the extended release dosage forms, optionally, coating of extended release dosage forms.

In further embodiment, the process of preparation comprises (a) compacting and milling pharmaceutically active agent and at least one pharmaceutically acceptable excipient to form compacted material (b) screening the compacted material to provide uniform sized active agent cores (c) coating the sized active agent cores with at least one release controlling polymer (d) blending and lubrication of coated active agent cores, with at least one pharmaceutical acceptable active agent and (d) compression of the blend to produce the extended release dosage forms, optionally, coating of extended release dosage forms.

In another embodiment, the process of preparation comprises (a) compacting and milling pharmaceutically active agent and at least one pharmaceutically acceptable excipient to form compacted material (b) screening the compacted material to provide uniform sized active agent cores (c) coating the sized individual active agent cores with a sub-coating layer (d) coating the sub-coated active agent cores with at least one release controlling polymer (e) coating the active agent cores with at least one cushioning top coat (f) blending and lubrication of coated active agent cores with at least one pharmaceutical acceptable active agent, and (g) compression of blend to produce the extended release dosage forms, optionally, coating of extended release dosage forms.

In another embodiment, the present invention also relates to a process for preparing an multiple unit extended release dosage form of metoprolol succinate comprising (a) compacting and milling pharmaceutically active agent and at least one pharmaceutically acceptable excipient to form compacted material (b) screening the compacted material to provide uniform sized active agent cores, (c) coating the active agent cores with at least one release controlling polymer, and (d) compression of coated active agent cores to produce the extended release dosage forms, optionally, coating of extended release dosage forms.

In a further embodiment, the present invention also relates to a process for preparing an multiple unit extended release dosage form of metoprolol succinate comprising (a) compacting and milling pharmaceutically active agent and at least one pharmaceutically acceptable excipient to form compacted material (b) screening the compacted material to provide uniform sized active agent cores, (c) coating the sized active agent cores with a sub-coating layer (d) coating the sub-coated active agent cores with at least one release controlling polymer, (e) coating the coated active agent cores with at least one cushioning top coat, (e) compression of blend to produce the extended release dosage forms, optionally, coating of extended release dosage forms.

In one embodiment, the bulk density of the active agent cores is not less than 0.3 g/ml. In one embodiment, the bulk density of the active agent cores is not less than 0.4 g/ml. In one embodiment, the bulk density of the active agent cores is not less than 0.5 g/ml.

In a further embodiment is provided use of the modified release compositions of the present invention for the prevention, treatment, management or mitigation of various disease conditions or disorders depending on the pharmaceutically active agent employed.

In another embodiment, the present invention also relates to extended release compositions comprising at least one second pharmaceutically active agent in addition to at least one pharmaceutically active agent present in the compositions. Such a second active agent includes, but is not limited to, the list of pharmaceutically active agents discussed above under pharmaceutically active agents. In a further embodiment, the second active agent may be delivered in an immediate or extended release manner. In one embodiment of the present invention, the multiple unit extended release dosage form comprises same or different pharmaceutically active agents. In another embodiment of the present invention, the active agents in the composition of the present invention can be delivered at different release rate and profiles as desired.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed, but that the invention will include all embodiments falling within the scope thereof. Details of the present invention, including its objects and advantages, are provided in the non-limiting exemplary illustrations below.

### Example 1: Metoprolol Succinate Extended Release Composition

| Ingredient | Mg/unit |
|---|---|
| Metoprolol succinate | 190 |
| Colloidal silicon dioxide | 2 |
| Magnesium stearate | 1 |
| Hydroxypropylmethyl | 21.5 |
| cellulose | |
| Active agent core | 214.5 |

Sub coating

| | |
|---|---|
| Active agent core | 214.5 |
| Ethylacrylate | 17 |
| methymethacrylate | |
| copolymer | |
| Sub coated active agent core | 231.5 |

Extended release coating

| | |
|---|---|
| Sub coated active agent core | 231.5 |
| Ethyl cellulose | 57 |
| Hydroxypropylmethyl | 19 |
| cellulose | |
| Triacetin | 12 |
| Talc | 38 |
| IPA | q.s |
| Purified water | q.s |
| Modified release units | 357.5 |

Compression

| | |
|---|---|
| Modified release units | 357.5 |
| Talc | 10 |
| Copovidone | 15 |
| Microcrystalline Cellulose | 238 |
| Croscarmellose sodium | 30 |
| Colloidal silicon dioxide | 8 |
| Sodium stearyl fumarate | 7 |
| Tablet | 665.5 |

Procedure:

Metoprolol succinate, colloidal silicon dioxide, magnesium stearate and hydroxypropylmethyl cellulose were sieved through mesh screen. The screened components were placed into blender and blending was carried out. The blend was further compacted and milled to get compacted active agent core of desired sieve fraction. Sub coating of compacted active agent core was carried out using ethylacrylate methymethacrylate copolymer. These sub coated pellets were further coated using ethyl cellulose and hydroxypropylmethyl cellulose to form modified release units. Modified release units were mixed with copovidone, microcrystalline cellulose, croscarmellose sodium, talc, sodium stearyl fumarate and colloidal silicon dioxide. The lubricated blend was compressed to obtain tablets.

### Example 2: Metoprolol Succinate Extended Release Composition

| Ingredient | Mg/unit |
|---|---|
| Metoprolol succinate | 190 |
| Colloidal silicon dioxide | 2 |
| Magnesium stearate | 1 |
| Hydroxypropylmethyl | 21.5 |
| cellulose | |
| Active agent core | 214.5 |

Sub coating

| | |
|---|---|
| Active agent core | 214.5 |
| Ethylacrylate | 17 |
| methymethacrylate | |
| copolymer | |
| Sub coated active agent core | 231.5 |

Extended release coating

| | |
|---|---|
| Sub coated active agent core | 231.5 |
| Ethyl cellulose | 57 |
| Hydroxypropylmethyl | 19 |
| cellulose | |
| Triacetin | 12 |
| Talc | 38 |
| IPA | q.s |
| Purified water | q.s |
| Modified release units | 357.5 |

PEG coating

| | |
|---|---|
| Modified release units | 357.5 |
| Polyethylene glycol | 34.5 |
| Purified water | q.s |
| PEG coated units | 392 |

Compression

| | |
|---|---|
| PEG coated units | 392 |
| Talc | 10 |
| Copovidone | 15 |
| Microcrystalline Cellulose | 238 |
| Croscarmellose sodium | 30 |
| Colloidal silicon dioxide | 8 |
| Sodium stearyl fumarate | 7 |
| Tablet | 700 |

Film Coating

| | |
|---|---|
| Core tablets | 700 |
| Opadry White | 25 |
| Purified water | q.s |
| Film coated tablet | 725 |

Procedure:

Metoprolol succinate, colloidal silicon dioxide, magnesium stearate and hydroxypropylmethyl cellulose were sieved through mesh screen. The screened components were placed into blender and blending was carried out. The blend was further compacted and milled to get compacted active agent core of desired sieve fraction. Sub coating of compacted active agent core was carried out using ethylacrylate methymethacrylate copolymer. These sub coated pellets were further coated using ethyl cellulose, hydroxypropylmethyl cellulose, triacetin, talc, to form modified release units.

Modified release units were coated with cushioning top coat of polyethylene glycol. These PEG coated units were blended with extragranular granules of talc, copovidone, microcrystalline cellulose, croscarmellose sodium, and colloidal silicon dioxide. This blend was lubricated with sodium stearyl fumarate. The lubricated blend was compressed to obtain tablets. Tablets were film coated with Opadry White to obtain Film coated tablet.

### Example 3: Diltiazem Hydrochloride Extended Release Composition

| Ingredient | Mg/unit |
|---|---|
| Diltiazem hydrochloride | 120 |
| Colloidal silicon dioxide | 2 |
| Magnesium stearate | 1 |
| Hydroxypropylmethyl | 20 |
| cellulose | |
| Active agent core | 143 |

Sub coating

| | |
|---|---|
| Active agent core | 143 |
| Ethylacrylate | 15 |
| methymethacrylate | |
| copolymer | |
| Sub coated active agent core | 158 |

Extended release coating

| | |
|---|---|
| Sub coated active agent core | 158 |
| Trimethylammonioethyl | 55 |
| methacrylate | |
| copolymer | |
| Hydroxypropylmethyl | 15 |
| cellulose | |
| Triacetin | 10 |
| Talc | 35 |
| IPA | q.s |
| Purified water | q.s |
| Modified release units | 273 |

Compression

| | |
|---|---|
| Modified release units | 273 |
| Talc | 10 |
| Copovidone | 15 |
| Microcrystalline Cellulose | 238 |
| Croscarmellose sodium | 30 |
| Colloidal silicon dioxide | 8 |
| Sodium stearyl fumarate | 7 |
| Tablet | 581 |

Procedure:

Diltiazem, colloidal silicon dioxide, magnesium stearate and hydroxypropylmethyl cellulose were sieved through mesh screen. The screened components were placed into blender and blending was carried out. The blend was further compacted and milled to get compacted active agent core of desired sieve fraction. Sub coating of compacted active agent core was carried out using Ethylacrylate methymethacrylate copolymer. These sub coated pellets were further coated using Trimethylammonioethyl methacrylate copolymer and hydroxypropylmethyl cellulose to form modified release units. Modified release units were mixed with copovidone, microcrystalline cellulose, croscarmellose sodium, talc, colloidal silicon dioxide and sodium stearyl fumarate. The lubricated blend was compressed to obtain tablets.

### Example 4: Diltiazem Hydrochloride Extended Release Composition

| Ingredient | Mg/unit |
|---|---|
| Diltiazem hydrochloride | 120 |
| Colloidal silicon dioxide | 2 |
| Magnesium stearate | 1 |
| Hydroxypropylmethyl | 20 |
| cellulose | |
| Active agent core | 143 |

Sub coating

| | |
|---|---|
| Active agent core | 143 |
| Ethylacrylate | 15 |
| methymethacrylate | |
| copolymer | |
| Sub coated active agent core | 158 |

Extended release coating

| | |
|---|---|
| Sub coated active agent core | 158 |
| Trimethylammonioethyl | 55 |
| methacrylate | |
| copolymer | |
| Hydroxypropylmethyl | 15 |
| cellulose | |
| Triacetin | 10 |
| Talc | 35 |
| IPA | q.s |
| Purified water | q.s |
| Modified release units | 273 |

PEG coating

| | |
|---|---|
| Modified release units | 273 |
| Polyethylene glycol | 44 |
| Purified water | q.s |
| PEG coated units | 317 |

Compression

| | |
|---|---|
| PEG coated units | 317 |
| Talc | 10 |
| Copovidone | 15 |
| Microcrystalline Cellulose | 238 |
| Croscarmellose sodium | 30 |
| Colloidal silicon dioxide | 8 |
| Sodium stearyl fumarate | 7 |
| Tablet | 625 |

Film Coating

| | |
|---|---|
| Core tablets | 625 |
| Opadry White | 25 |
| Purified water | q.s |
| Film coated tablet | 650 |

Procedure:

Diltiazem hydrochloride, colloidal silicon dioxide, magnesium stearate and hydroxypropylmethyl cellulose were sieved through mesh screen. The screened components were placed into blender and blending was carried out. The blend was further compacted and milled to get compacted active agent core of desired sieve fraction. Sub coating of compacted active agent core was carried out using ethylacrylate methymethacrylate copolymer. These sub coated pellets were further coated using trimethylammonioethyl methacrylate copolymer, hydroxypropylmethyl cellulose, triacetin, talc to form modified release units. Modified release units were coated with polyethylene glycol to form PEG coated units. Extra granular granules of talc, copovidone, microcrystalline cellulose, croscarmellose sodium, and colloidal silicon dioxide were blended with PEG coated units. The blend was lubricated with sodium stearyl fumarate. The lubricated blend was compressed to obtain tablets. Tablets were film coated with Opadry White to obtain film coated tablets.

We claim:

1) An extended release pharmaceutical composition comprising plurality of modified release units wherein each unit comprises of an active agent core comprising at least one pharmaceutically active agent and at least one pharmaceutically acceptable excipient, substantially coated with at least one release-controlling agent.

2) The composition of claim 1 wherein the active agent is a psychostimulant, an antihistamine, an expectorant or a mucolytic, an anti-tussive agent, a serotonin reuptake inhibitor, a norepinephrine reuptake inhibitor, a sympatholytic, an antipsychotic, an antimuscarinic, a urinary antispasmodic, a PDE5 inhibitor, an anti-Alzheimer's agent, an analgesic, a decongestant, an analeptic agent, an anesthetic agent, an anti-asthmatic, an anti-arthritic agent, an anti-cancer agent, an anti-cholinergic agent, an anti-convulsant agent, an anti-depressant agent, an antidiabetic, an anti-helminthic agent, an anti-diarrheal agent, an anti-epileptic, an anti-hyperlipidemic agent, an antihypertensive agent, an antihypotensive agent, an anti-infective agent, an anti-inflammatory agent, a non-steroidal anti-inflammatory agent, an anti-emetic, an anti-migraine agent, an anti-neoplastic agent, an anti-tubercular agent, an antibiotic, an antacid, an antiulcer agent, an anti-Parkinsonism drug, an anti-pruritic agent, an antipsychotic agent, an anti-pyretic agent, an anti-spasmodic, an anti-viral agent, an anxiolytic agent, an appetite suppressant, an attention deficit hyperactivity disorder agent, a cardiovascular agent, a calcium channel blocker, an antianginal agent, a central nervous system agent, a beta-blocker, an antiarrhythmic agent, a bronchodilator, a central nervous system stimulant, a diuretic, a genetic material, a hormonolytics; a hypnotic, a hypercalcemic, a hypoglycemic agent, a immunosuppressive agent, a beta-agonist, a narcotic antagonist, nicotine, a nutritional agent, a parasympatholytic, a peptide drug, an antihemorrhoidal, a psychostimulant, a psychotropic, a mucolytic, a sedative, a laxative, a vitamin, a sialagogue, a steroid, a sympathomimetic, a tranquilizer, a vasodilator, or any combination thereof.

3) The composition of claim 2 wherein the active agent is in the form of free base, free acid, pharmaceutically acceptable salt, prodrug, active metabolite, polymorph, solvate, hydrate, enantiomer, optical isomer, tautomer or racemic mixture thereof, or any combination thereof.

4) The composition of claim 2 wherein the active agent is amphetamine, amphetaminil, atomoxetine, dexmethylphenidate, dextroamphetamine, dextromethamphetamine, cyclobenzaprine, propranolol, fencamfamine fenethylline, lisdexamfetamine, methylphenidate, mesocarb, pemoline, pipradrol prolintane, dimenhydrinate, diphenhydramine, chlorpheniramine, brompheniramine, dexchlorpheniramine, hydroxyzine, dexbrompheniramine, fexofenadine, terfenadine, cetirizine, levocetirizine, ambroxol, bromhexine, carbocisteine, domiodol, guaifenesin, codeine, dextromethorphan, hydrocodone, clovoxamine, desvenlafaxine, naltrexone, duloxetine, levomilnacipran, eclanamine, milnacipran, sibutramine, venlafaxine, alaproclate, citalopram, escitalopram, femoxetine, fluoxetine, fluvoxamine, indalpine, ifoxetine litoxetine, omiloxetine, panuramine, paroxetine, pirandamine, seproxetine, sertraline, zimelidine, clonidine, guanfacine, methyldopa, iloperidone, ocaperidone, paliperidone, risperidone, lurasidone, perospirone, revospirone, tiospirone, ziprasidone, darifenacin, emepronium, fesoterodine, flavoxate, imidafenacin, meladrazine, mirabegron, oxybutynin, propiverine, solifenacin, terodiline, esomeprazole, omeprazole, pantoprazole, lansoprazole, dexlansoprazole, tolterodine, trospium chloride, acetildenafil, aildenafil, avanafil, icariin, lodenafil, mirodenafil, nitrosoprodenafil, sildenafil, sulfoaildenafil, tadalafil, udenafil, vardenafil, memantine, neramexane (1, 3, 3, 5, 5-pentamethylcyclohexan-1-amine), donepezil, tacrine, rivastigmine, galantamine, physostigmine, neostigmine, Huperzine A, icopezil (CP-118954; 5,7-dihydro-3-[2-[1-(phenylmethyl)-4-piperidinyl]ethyl]-6H-pyrrolo-[4,5-f]-1,2-benzisoxazol-6-onemaleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl) piperidin-4-yl]-1-(2,3,4,5-tetrahydro-1H-1-benzazepin-8-yl)-1-propane fumarate), metrifonate (T-588; (-)-R-α-[[2-(dimethylamino)ethoxy]methyl] benzo [b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f] oxepine-10-methanamine), SDZ-220-581 ((S)-α-amino-5-(phosphonomethyl)-[1,1′-biphenyl]-3-propionic acid), tarenflurbil, tramiprosate, clioquinol, aspirin, codeine, morphine, dihydromorphone, oxycodone, hydrocodone, phenylephrine, pseudoephedrine, theophylline, phenobarbital sodium, phenytoin sodium, valproate sodium barbiturates, amylobarbitone sodium, butabarbital sodium, secobarbital sodium, metformin; phenytoin, meprobamate, nitrezepam, oxcarbazepine, methyldopa, captopril, naproxen, diclofenac, indomethacin, ibuprofen, sulindac, meclofenamate sodium, tolmetin sodium, metoclopramide, tetracyclines, atropine, scopolamine, albuterol, ethacrynic acid, bendrofluazide, nifedipine, papaverine, diltiazem, or nicardirine or any combination thereof.

5) The composition of claim 4 wherein the active agent is metoprolol succinate.

6) The composition of claim 1 wherein the pharmaceutically acceptable excipient is diluent, binder, glidant, lubricant, anti-adherent or any combinations thereof.

7) The composition of claim 1 wherein the active agent core is in the form of dry granules or compacted granules.

8) The composition of claim 7 wherein the active agent core is prepared by roller compaction process.

9) The composition of claim 1 wherein the release-controlling agent is polymeric release controlling agent, non-polymeric release controlling agent or any combination thereof.

10) The composition of claim 9 wherein the release-controlling agent is pH-dependent release controlling agent, pH-independent release controlling agent or any combination thereof.

11) The composition of claim 9 wherein the polymeric release controlling agent is a cellulose derivative, an acrylic acid derivative, a maleic acid derivative, polymer and copolymer, a vinyl derivative, polymer and copolymer or any combination thereof.

12) The composition of claim 9 wherein the polymeric release controlling agent is ethyl cellulose, hydroxypropylmethyl cellulose, hydroxypropylmethylcellulose acetate succinate, hydroxypropylmethyl cellulose phthalate, hydroxymethylethylcellulose phthalate, cellulose acetate phthalate, cellulose acetate succinate, cellulose acetate maleate, cellulose acetate trimelliate, cellulose benzoate phthalate, cellulose propionate phthalate, methyl cellulose phthalate, carboxymethylethylcellulose, ethylhydroxyethylcellulose phthalate, styrene/acrylic acid copolymer, methyl acrylate/methacrylic acid copolymer, butyl acrylate/styrene/acrylic acid copolymer, methacrylic acid polymers and copolymers, polyacrylates, methacrylate polymers and copolymers, copolymer formed from monomers selected from methacrylic acid, methacrylic acid esters, acrylic acid and acrylic acid esters, copolymer formed from monomers selected from butyl methacrylate, (2-dimethylaminoethyl)methacrylate and methyl methacrylate, copolymer formed from monomers selected from ethyl acrylate, methyl methacrylate and trimethylammonioethyl methacrylate chloride, copolymers of acrylate and methacrylates with/without quarternary ammonium group in combination with sodium carboxymethylcellulose, dimethylaminoethylmethacrylate copolymer, trimethylammonioethyl methacrylate copolymer, ethylacrylate methylmethacrylate copolymer, methacrylic acid methyl methacrylate copolymer, methacrylic acid ethyl acrylate copolymer, vinylacetate/maleic acid anhydride copolymer, styrene/maleic acid anhydride copolymer, styrene/maleic acid monoester copolymer, vinylmethylether/maleic acid anhydride copolymer, ethylene/maleic acid anhydride copolymer, vinylbutylether/maleic acid anhydride copolymer, acrylonitrile/methyl acrylate/maleic acid anhydride copolymer, butyl acrylate/styrene maleic acid anhydride copolymer, polyvinylacetate, copolymers of vinyl pyrrolidone, polyvinyl alcohol, copolymers of polyvinyl alcohol, mixture of polyvinyl acetate and polyvinylpyrrolidone, polyvinyl alcohol phthalate, polyvinylacetal phthalate, polyvinyl butylate phthalate, polyvinylacetoacetal phthalate, or any combination thereof.

13) The composition of claim 9 wherein the polymeric release-controlling agent is ethyl cellulose.

14) The composition of claim 1 wherein the active agent cores are coated with at least one sub-coat prior to coating with at least one release-controlling agent.

15) The composition of claim 1 wherein the active agent core substantially coated with release-controlling agent comprises at least one cushioning top coat.

16) The composition of claim 15 wherein the cushioning top coat comprises at least one hydrophilic polymer.

17) The composition of claim 16 wherein the hydrophilic polymer is polyethylene glycol.

18) The composition of claim 1 wherein the composition further comprises at least one pharmaceutically acceptable excipient.

19) The composition of claim 18 wherein the pharmaceutically acceptable excipient is a binder, diluent, disintegrant, superdisintegrant, flavor, sweetener, colorant, salivating agent, surfactant, souring agent, viscolizer, solubilizer, stabilizer, suspending agent, preservative, cosolvent, anti-caking agent, buffer, glidant, lubricant or any combination thereof.

20) The composition of claim 1 wherein the extended release pharmaceutical composition is in the form of tablets, capsules, mini tablets, orally disintegrating tablets, granules, or dispersible tablets.

21) The composition of claim 1 wherein the composition further comprises at least one second active agent.

22) The composition of claim 21 wherein the second active agent is delivered in an immediate release or extended release manner.

23) The process for preparing compositions of claim 1 comprising (a) dry granulating/compacting and milling pharmaceutically active agent and at least one pharmaceutically acceptable excipient to form dry granules or compacted granules (b) screening the compacted/dry granulated material to provide uniform sized active agent cores (c) coating the active agent cores with at least one release controlling polymer (d) blending and lubrication of coated active agent cores with at least one pharmaceutically acceptable excipient, and (e) compression of the blend to produce the extended release dosage forms, optionally, coating of extended release dosage forms.
